(19) United States
(12) Patent Application Publication
(10) Pub. No.: US 2021/0085818 A1
(43) Pub. Date: Mar. 25, 2021

(54) Germicidal Treatment System and Method

(71) Applicant: Daniel Jones, Valencia, CA (US)

(72) Inventor: Daniel Jones, Valencia, CA (US)

(21) Appl. No.: 17/024,888

(22) Filed: Sep. 18, 2020

Related U.S. Application Data
(60) Provisional application No. 62/902,795, filed on Sep. 19, 2019.

(51) Int. Cl. A61L 11/00; A61L 2/10

(52) U.S. Cl. CPC A61L 11/00; A61L 2202/23; A61L 2202/11; A61L 2/10

(57) ABSTRACT

Embodiments described herein relate to a system and method for germicidally irradiating a waste receptacle or other receptacle that fosters microbial growth. A system for treating a waste receptacle may include a housing; a power source disposed about the housing for providing power; and a germicidal source energetically coupled to the power source for irradiating the waste receptacle. The waste receptacle may be a garbage disposal. The germicidal source may be an ultraviolet lamp. A method for treating a waste receptacle may include providing a housing; providing a power source disposed about the housing for providing power; and providing a germicidal lamp energetically coupled to the power source for irradiating the waste receptacle.

### CROSS-REFERENCE OF RELATED APPLICATIONS

The present disclosure claims priority to, and the benefit of, U.S. provisional application Ser. No. 62/902,795, titled Germicidal Treatment System and Method, and filed on Sep. 19, 2019, which is incorporated herein by reference in its entirety.

### BACKGROUND

Waste receptacles and other containers for trash or refuse are repositories for germs and harmful bacteria. Often, common household items used for storing or disposing of waste can also house such germs and bacteria. For example, garbage disposals are common household appliances typically used for disposing post-meal food products and other waste items. However, mold and other bacteria which may be harmful can grow in garbage disposals, resulting in unwanted microbial growth and foul odors.

### SUMMARY

Embodiments described herein relate to a system and method for germicidally treating a waste receptacle, such as a garbage disposal, for example, that fosters microbial growth. A system for treating a waste receptacle may include a housing; a power source disposed about the housing for providing power; and a germicidal source energetically coupled to the power source for irradiating the waste receptacle. The waste receptacle may be a garbage disposal. The germicidal source may be an ultraviolet lamp, such as a UV-C lamp, or a light emitting diode (LED) lamp. The housing may be configured to be removably coupled to the waste receptacle. A method for germicidally treating a waste receptacle may include providing a housing; providing a power source disposed about the housing for providing power; and providing a germicidal source energetically coupled to the power source for irradiating the waste receptacle.

### BRIEF DESCRIPTION OF THE DRAWINGS

FIG. 1 is a schematic block diagram illustrating an example of a general waste receptacle treatment system according to various embodiments of the invention.

FIG. 2 shows top, bottom and side views of a waste receptacle treatment system according to various embodiments of the invention.

### DETAILED DESCRIPTION

In the following description of various embodiments, reference is made to the accompanying drawings which form a part hereof and in which are shown by way of illustration specific embodiments in which the embodiments may be practiced. It is to be understood that other embodiments may be utilized, and structural changes may be made without departing from the scope of the various embodiments disclosed in the present disclosure.

Embodiments described herein generally relate germicidal treatment systems and methods. Embodiments described herein also generally relate to waste receptacle treatment systems and methods. Embodiments of the waste receptacle treatment system and method may include a germicidal source, a germicidal reflector, a germicidal source protector, and an electronic ballast. Embodiments of the invention may also include a safety shut-off, a rechargeable port, a water-tight seal for the re-charging port, an on indicator, an on-off switch and a plastic enclosure. According to embodiments of the present invention, the waste receptacle treatment system and method is designed to be placed on top of a common kitchen waste receptacle, similar to a sink stopper, to irradiate the internal cavity of the waste receptacle (for example, side walls, bottom, blades, and the like), killing mold and bacteria. It should be appreciated that waste receptacle treatment systems and methods are likewise applicable to other configurations, in addition to that described above.

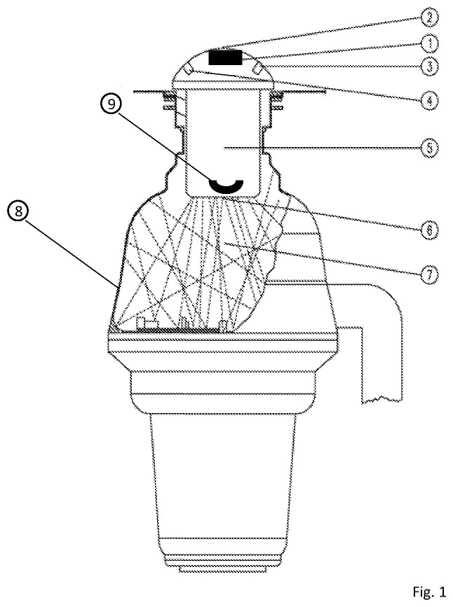

FIG. 1 is a schematic block diagram illustrating an example of a general waste receptacle treatment system according to various embodiments of the invention. In various embodiments, the waste receptacle treatment system 2 may include a germicidal source 9, a germicidal reflector 6, a housing 5, and a power source 1. Embodiments of the invention may also include various other elements such as an on-off switch 3 and a port 4. In some embodiments, the invention may also include a safety shut-off, a water-tight seal for the re-charging port, an on indicator, and a plastic enclosure. In the embodiment shown in FIG. 1, the waste receptacle treatment system 2 is disposed adjacent to a waste receptacle 8 such that the germicidal source 9 may irradiate with radiation 7 the internal cavity of the waste receptacle 8 and any components of the waste receptacle disposed within the internal cavity, such as the side walls, bottom, blades, and the like, for example, thereby killing mold and bacteria that may be the source of foul odors and disease. For example, as shown in FIG. 1, the waste receptacle treatment system 2 is displaced on a portion of the waste receptacle 8 that exposes the internal cavity of the waste receptacle 8. The waste receptacle 8 as shown in the embodiment of FIG. 1 is configured as a common garbage disposal, which is typically coupled to a kitchen sink having an opening on one of its surfaces designed specifically to interface with an opening to a cavity of waste receptacle and provides a convenient location for displacement of the waste receptacle treatment system 2.

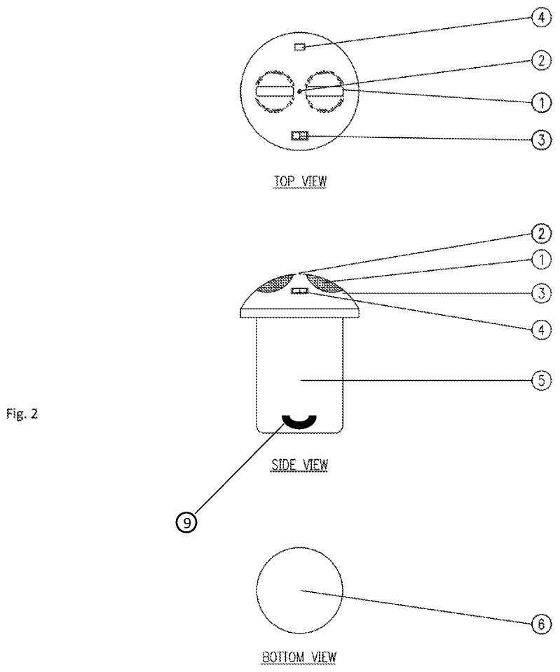

FIG. 2 shows top, bottom and side views of a waste receptacle treatment system 2 according to various embodiments of the invention. In various embodiments, the waste receptacle treatment system 2 may include a germicidal source 9, a germicidal reflector 6, a housing 5, and a power source 1. Embodiments of the invention may also include various other elements such as an on-off switch 3 and a port 4. The germicidal source 9 may be an ultra-violet lamp, such as a common UV-C lamp, a germicidal light emitting diode (LED) lamp, a low-pressure mercury vapor lamp and the like. The germicidal source 9 may be integrated with the power source 1 and the housing 5. The germicidal source 9 may radiate germicidal energy substantially at wavelengths in the range of 200-280 nm, which have shown to kill microbial growth, a source of foul orders. Other wavelengths may be chosen to address other applications, such as the elimination of various bacteria and viruses. The germicidal source reflector 6 may be made of quartz, plastic, glass or any other material capable of reflecting or directing the propagation of the germicidal source 9 radiation. The housing 5 may be made of plastic or metal. The housing 5 may be configured such that it is removably coupled to a waste receptacle. The power source 1 could be a battery, such as a lithium ion, alkaline or carbon zinc battery. The power source 1 may be rechargeable. Also, the power source 1 could be configured as an AC source such that the waste receptacle treatment system 2 is plugged into a common household electrical outlet. The port 4 may be configured as a port for recharging the power source 1 or configured as a power cable interface between the power source 1 and a common household electrical outlet. The on-off switch 3 may be coupled to the power source 1 and configured as a manual switch, such as a slide switch or push-button switch, allowing a user to turn the waste receptacle treatment system 2 on and off.

Embodiments of the invention may be used in a variety of ways. For example, the waste receptacle treatment system 2 may be removably coupled or positioned in a common kitchen garbage disposal such that the germicidal reflector 6 is exposed to an internal portion of the garbage disposal. The waste receptacle treatment system 2 may be turned on using the on-off switch 3, thereby engaging the power source 1 and providing power to the germicidal source 9. The radiation from the germicidal source 9 is then dispersed throughout the internal portion of the garbage disposal via the germicidal reflector 6, thereby eliminated foul odors, bacteria, viruses, and the like.

The embodiments disclosed herein are to be considered in all respects as illustrative, and not restrictive. The present disclosure is in no way limited to the embodiments described above. Various modifications and changes may be made to the embodiments without departing from the spirit and scope of the disclosure. Various modifications and changes that come within the meaning and range of equivalency of the claims are intended to be within the scope of the disclosure.

What is claimed is:

1. A system for treating a waste receptacle comprising:
a housing;
a power source disposed about the housing for providing power; and
a germicidal source energetically coupled to the power source for irradiating the waste receptacle.

2. The system of claim 1, wherein the germicidal source is an ultraviolet lamp.

3. The system of claim 2, wherein the ultraviolet lamp is a UV-C lamp.

4. The system of claim 1, wherein the germicidal source is an LED lamp.

5. The system of claim 1, wherein the housing is configured to be removably coupled to the waste receptacle.

6. A method for treating a waste receptacle comprising:
providing a housing;
providing a power source disposed about the housing for providing power; and
providing a germicidal source energetically coupled to the power source for irradiating the waste receptacle.

7. The method of claim 6, wherein the germicidal source is an ultraviolet lamp.

8. The method of claim 7, wherein the ultraviolet lamp is a UV-C lamp.

9. The method of claim 6, wherein the germicidal source is an LED lamp.

10. The method of claim 6, wherein the housing is configured to be removably coupled to the waste receptacle.
